(19) United States
(12) Patent Application Publication
(10) Pub. No.: US 2008/0119915 A1
(43) Pub. Date: May 22, 2008

(54) Spotless Tanning Support Device

(76) Inventor: Kristina M. McKay, Chicago, IL (US)

Correspondence Address: CARDINAL LAW GROUP, SUITE 2000, 1603 ORRINGTON AVENUE, EVANSTON, IL, 60201, US

(21) Appl. No.: 11/548,600

(22) Filed: Oct. 11, 2006

(51) Int. Cl. A61N 5/06

(52) U.S. Cl. 607/94

(57) ABSTRACT

Embodiments of the invention relate to a system and method for providing body support while tanning, using a tanning bed for example. In one embodiment, the method comprises positioning a support device on at least one surface (i.e. a UV emitting surface for example) of the tanning bed. A tailbone region of a user is positioned on the support device, elevating the user so that only the user's feet and shoulders contact the surface while reclining in the tanning bed. Ultraviolet light is applied to the user, initiating the tanning session. In one embodiment, the invention relates to a support device for use with a tanning bed or other warm surface. The support device comprises first, second and third surfaces. The first surface defines a first peripheral edge; the second surface is opposite the first surface and defines a second peripheral edge; the third surface defines two opposing peripheral edges, at least one of the two opposing peripheral edges contacting the first peripheral edge and the other contacting the second peripheral edge. A recess is formed in at least one of the first and second surfaces and is configured to be placed under a user's tailbone.

### FIELD OF THE INVENTION

One embodiment of the invention relates to a system and method for obtaining a tan using a tanning bed. More specifically, one embodiment relates to a system and method of obtaining a tan using a support pillow with a tanning bed.

### BACKGROUND OF THE INVENTION

Tanning booths or beds making use of artificial ultra violet (UV) light are very popular, especially in the winter months when a natural tan can not be readily acquired and particularly in those areas where the summer tanning season is relatively short. Many tanning operators own a number of tanning beds for rent to the public.

A typical tanning bed has an upper tanning unit and a lower tanning unit connected by hinges so that the upper unit can be opened and a closed in a manner analogous to a clamshell. In the open position, a person can enter the apparatus to lie down on the lower tanning unit. In the closed position, the units form an internal tanning chamber where the user is partially surrounded by tanning lamps. The upper unit has a row of lamps that are intended to tan the upwardly facing parts of the person, and the lower unit has a row of lamps that are intended to tan the downwardly facing parts of the person.

One problem that is often encountered in prior art devices is uneven tanning around the user's buttocks and sides. As the user's buttocks are pressed against the bed surface (one or more UV emitting surfaces for example), rounded areas of skin are pressed against the tanning bed surface producing a substantially flat surface-to-surface contact. As such, the user may experience uneven tanning, spotting, or even burns due to the uneven UV light exposure in comparison to the rest of the body. The user may alternatively have to resort to wearing an undergarment or other protective device, which may result in an uneven tan, or sometimes risk getting overexposed to the UV rays.

Various pillows and cooling devices are known in the art. U.S. Pat. No. 3,312,987 to Emery, issued Apr. 11, 1967 discloses a U-shaped pillow. U.S. Pat. No. 4,858,259 to Simmons et al., issued Aug. 22, 1989 discloses a therapy pillow with removable therapeutic pack. U.S. Pat. No. 4,887,326 to O'Brien et al., issued Dec. 19, 1989 discloses a suboccipital pillow. U.S. Pat. No. 5,344,437 to Pistay, issued Sep. 6, 1994 discloses a massaging therapeutic pillow with removable ice pack. U.S. Pat. No. 5,545,199 to Hudson, issued Aug. 13, 1996 discloses a hot and cold therapeutic pillow. U.S. Pat. No. 5,916,088 to Gueli, issued Jun. 29, 1999 discloses a pillow/headrest designed to keep a user cool (using a cooling packs or gels). U.S. Pat. No. 6,782,572 to Jones, issued Aug. 31, 2004 discloses a multi-use pillow adapted to be mounted to a tanning bed using straps for example.

Such disclosed pillows and cooling devices are generally adapted to keep a user comfortable and cool while in a reclining position. Such known pillows and cooling devices are not adapted to support the user while tanning, preventing possible injury while simultaneously promoting even tanning.

It would, therefore, be desirable to provide a system and method that overcomes the above disadvantages, providing a more homogeneous tan in a tanning booth that overcomes the aforementioned and other disadvantages.

### SUMMARY OF THE INVENTION

One aspect of the invention relates to a method for tanning using a tanning bed. One embodiment of the method comprises positioning a support device on at least one surface (i.e. a UV emitting surface for example) of the tanning bed. A tailbone region of a user is positioned on the support device, elevating the user so that only the user's feet and shoulders contact the surface while reclining in the tanning bed. Ultraviolet light is applied to the user, initiating the tanning session.

The tanning method comprises receiving the user's buttocks in a recess formed in the support device. The tanning method comprises terminating the tanning session. In one embodiment, terminating the tanning session comprises terminating the ultraviolet light and opening the tanning bed. The method may further comprise removing the support device from the UV emitting surface and either sterilizing or disposing of it.

Another aspect of the invention relates to a tanning bed system. The tanning bed system comprises a tanning bed and a body support portion. The body support portion comprises first, second and third surfaces and a recess. The first surface defines a first peripheral edge while the second surface comprises a second surface opposite the first surface, the second surface defining a second peripheral edge. The third surface defines two opposing peripheral edges, at least one of the two opposing peripheral edges contacting the first peripheral edge and the other of the two opposing peripheral edges contacting the second peripheral edge. The recess is formed in at least one of the first and second surfaces and configured to be placed under a user's tailbone region.

In one embodiment, the recess is formed in the first surface and at least one of the first, second and third surfaces are substantially planar. The material of the support device is configured not to compress by more than half when a force of less than about 500 pounds is applied thereto. In one embodiment, the material is either substantially transparent or opaque to ultraviolet light. More specifically, the material is selected from a group consisting of plastic, porous material, foam, silicone, and rubber. The material is configured to be sterilized and reused or disposed of (i.e., thrown away)

Another aspect of the invention relates to a support device for use with a tanning bed. The support device comprises first, second and third surfaces. The substantially planar first surface having at least two sets of opposing ends, the first surface defining a first peripheral edge. The substantially planar second surface is opposite the first surface and has at least two sets of opposing ends and defines a second peripheral edge, where the substantially planar second surface is configured to contact at least one UV emitting surface of the tanning bed. The third surface defines two opposing peripheral edges, one of the two opposing peripheral edges contacting the first peripheral edge and the other of the two opposing peripheral edges contacting the second peripheral edge. A recess is formed in the first surface and configured to be placed under a user's tailbone region, elevating the user such that only the user's feet and shoulders contact the UV emitting surface while reclining or lying supine in the tanning bed. Each of the two sets of opposing edges are about 4 inches in length and the two opposing peripheral edges of the third surface are spaced about 2 inches apart.

The foregoing and other features and advantages of the invention will become further apparent from the following detailed description of the presently preferred embodiment, read in conjunction with the accompanying drawings. The drawings are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 depicts a front perspective view of a support device or pillow in accordance with one embodiment of the invention;

FIG. 2 depicts a back perspective view of the support device or pillow of FIG. 1 in accordance with one embodiment of the invention; and

FIG. 3 depicts a side elevational view of the support device of FIGS. 1 and 2 in accordance with one embodiment of the invention.

FIGS. 4-6 depict perspective views of support devices or pillows in accordance with other embodiments of the present invention;

FIG. 7 depicts a perspective view of a tanner reclining or lying supine in a tanning bed using a support device or pillow similar to that illustrated in FIGS. 1-6 in accordance with one embodiment of the present invention; and

FIG. 8 depicts a flow diagram illustrating one method of using a support device or pillow in accordance with one embodiment of the invention.

Throughout the various figures, like reference numbers refer to like elements.

### DETAILED DESCRIPTION OF PRESENTLY PREFERRED EMBODIMENTS

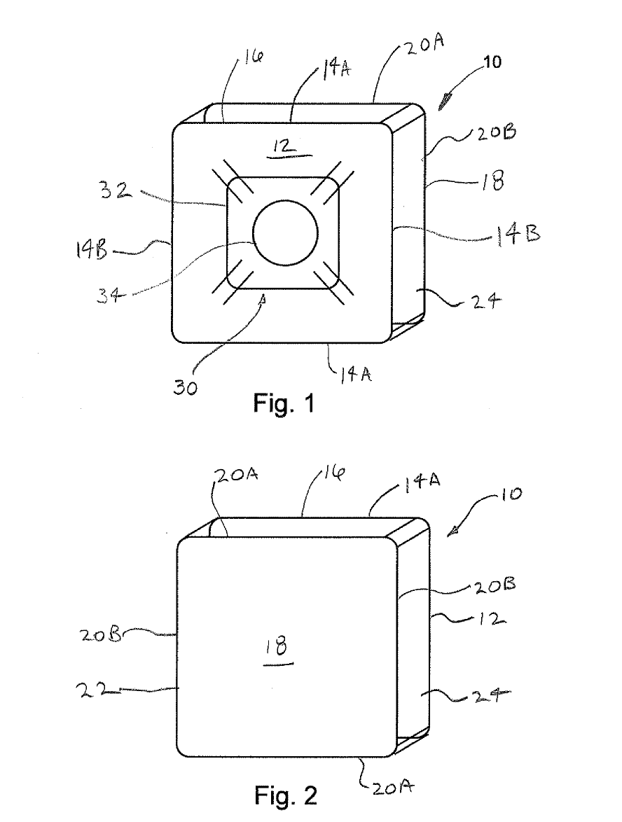

FIGS. 1-3 illustrate one embodiment of a body support device or pillow, generally designated 10, adapted to by used by a user in conjunction with a tanning bed or other warm surface. In FIGS. 1-3, device 10 is shown as a rounded square ranging between 2 and 6 inches in length and width and between 1 and 4 inches in depth. The illustrated embodiment is 4 inches by 4 inches (length-by-width) versus 2 inches deep. Those skilled in the art will appreciate that that the size and shape of the device 10 is not limited to the examples provided herein.

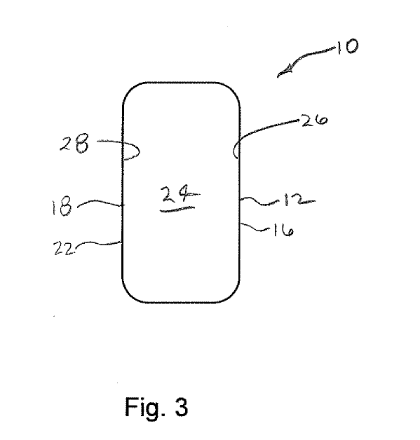

FIG. 1 depicts a perspective view of the front side of device 10; the device 10 comprising substantially planar first surface or portion 12 having two sets of opposing ends 14A and 14B and defining peripheral edge 16. Device 10 further comprises substantially planar second surface or portion 18 defining two sets of opposing ends 20A and 20B (opposite opposing ends 14A and 14B respectively) defining peripheral edge 22 (best viewed in FIG. 2). A third surface or portion 24 is shown adjacent to and contacting first and second surfaces 12 and 18. In the illustrated embodiment, third surface 24 defines two opposing peripheral edges 26 and 28 (best viewed in FIG. 3) where peripheral edges 26 and 28 are adjacent to and contact first and second peripheral edges 16 and 18 respectively.

FIG. 1 further illustrates that first surface 12 defines at least one recess 30 having a predetermined shape, size and in a particular area of the first surface 12. In FIG. 1, recess 30 comprises a stepped or multi-staged recess comprising a generally rectangular first stage 32 and a rounded second stage 34 formed approximately in the middle of first surface 12, although other arrangements are contemplated (i.e., a single stage and/or rectangle, oval and multi-sided shapes are contemplated) placed differently in the first surface 12. In one embodiment, the recess 30 comprises an aperture that extends through the device 10.

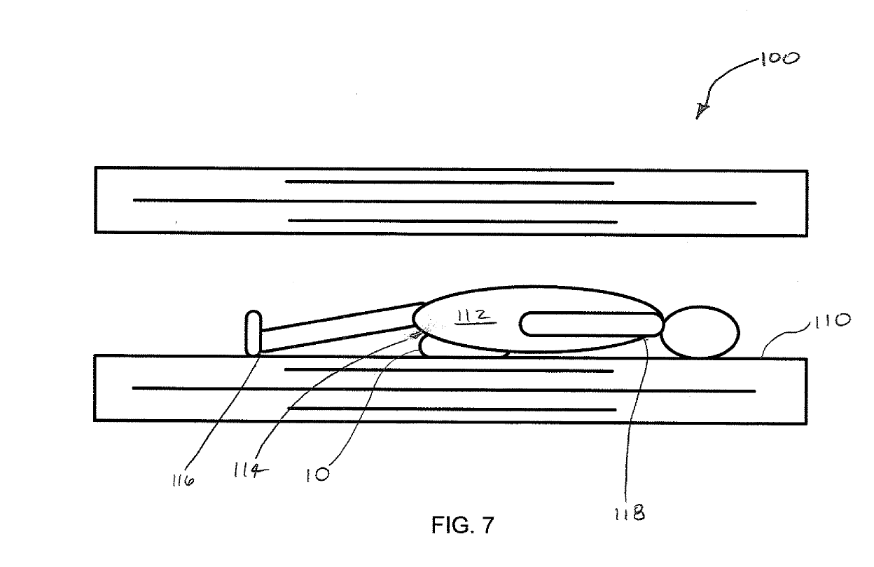

In at least one embodiment, the recess 30 is configured to be placed under the user's tailbone (e.g., coccyx) during a tanning session. Recess 30 fits or conforms to the user's buttocks, wherein the user is supported such that only the user's shoulders and feet are in contact with the tanning bed or other warm surface (best viewed in FIG. 7) while reclining or lying supine.

FIG. 2 illustrates the substantially planar second surface 18 configured to contact a surface of a tanning bed or other warm surface, enabling device 10 to be attached to the tanning bed by frictional forces for example. Alternatively, device 10 may include Velcro® adhesive, strong or weak adhesives, straps, etc, to secure the device 10 to the tanning bed. It is also contemplated that, in at least one embodiment, second surface 18 may define a second recess similar to, and a mirror image of, recess 20, such that the device 10 is reversible.

FIG. 3 illustrates third surface or portion 24. In the illustrated embodiment, third surface 24 defines two opposing peripheral edges 26 and 28 (best viewed in FIG. 3) where peripheral edges 26 and 28 are adjacent to and contact first and second peripheral edges 16 and 18 respectively. As illustrated, third surface 24 is formed such that the two opposing edges 26 and 28 are about 2 inches apart. However, it should be appreciated by one skilled in the art that a third surface having a varying width is contemplated, such that one end of the device is thicker than the other and the support device 10 has a wedge-like shape when viewed from the side.

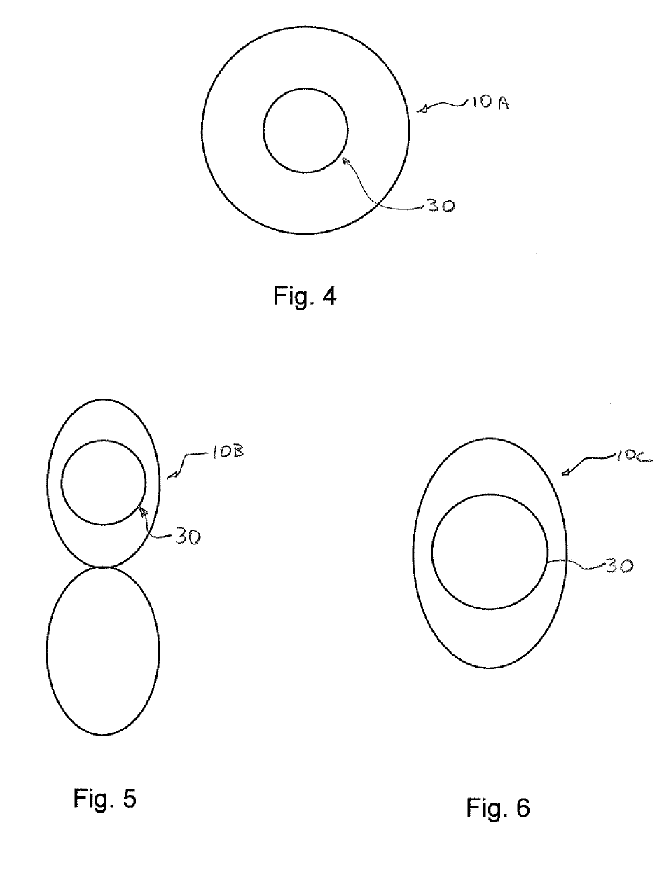

Other embodiments of the support device having different shapes are illustrated in FIGS. 4-6. For example, device 10 illustrated in FIG. 1 is a square. However, as illustrated in FIG. 4, the device 10A is round or a sphere, the device 10B illustrated in FIG. 5 is oblong or a figure eight, while the device 10C of FIG. 6 is an oval. Those skilled in the art will recognize that the size and shape of the device 10 may vary depending on the physiques and comfort of the user and are not limited to the descriptions and illustrations provided herein.

In one embodiment, the device 10 is comprised of or manufactured from a material that allows a portion of ultraviolet energy (e.g., either or both UV-A and UV-B rays) to transmit there through (i.e., the material is transparent, substantially transparent, substantially opaque or opaque [i.e., blocks] UV light). Device 10 may comprise a resilient (e.g., compressible) material such as, plastic, porous material, foam, silicone, rubber, and the like. In one embodiment, the material is formed such that it does not compress by more than half its width when a force of less than 500 pounds is applied.

The constituent material of the device 10 provides comfort to the user during the tanning session, which typically lasts about 15-45 minutes. The use of compressible materials includes, but is not limited to, softer plastics, porous materials, and foams generally represent suitable materials as they are generally inexpensive, hygienic, and reusable. Those skilled in the art will appreciate that a myriad of other materials (including disposable materials) may also be adapted for use with the present invention.

FIG. 7 illustrates one embodiment of the device 10 used with a tanning bed or other warm surface 100. Device 10 is positioned on the surface 110 of tanning bed 100, between surface 110 and a user 112. Device 10 is positioned on surface 110 and approximately under the user's tailbone 114 (e.g., coccyx), such that the user's buttock's is received in the recess thereof. This arrangement elevates the user 112 off of the tanning bed surface 110 while reclining or lying in a supine position, such that user 112 contacts the tanning bed surface 110 predominantly at the feet or ankles 116 and shoulder blades or head 118. As the user 112 receives a treatment of ultraviolet light, skin melonocytes produce a brown pigment commonly referred to as tanning. After the tanning session, the device 110 may be easily removed, cleaned, stored or disposed of.

In one embodiment, during UV light transmission, it is preferable that the device 10 does not exceed room temperature by a great margin. Excessive temperature may likely cause discomfort to the user. As such, numerous materials, such as clear plastics, are contemplated which are capable of transmitting UV light without storing a large amount of heat (i.e., becoming too hot to use). Other measures, such as adding a cooling pad or gel, fan, or other cooling technology to ensure a cool device 10 are also contemplated.

As illustrated in FIG. 1, the device 10 includes recess 30 formed therein. Recess 30 not only allows the user 112 to comfortable use the tanning bed 100, but additionally enables the user 112 to align himself/herself in the proper tanning position (e.g., by positioning oneself left, right, up, down, etc.)

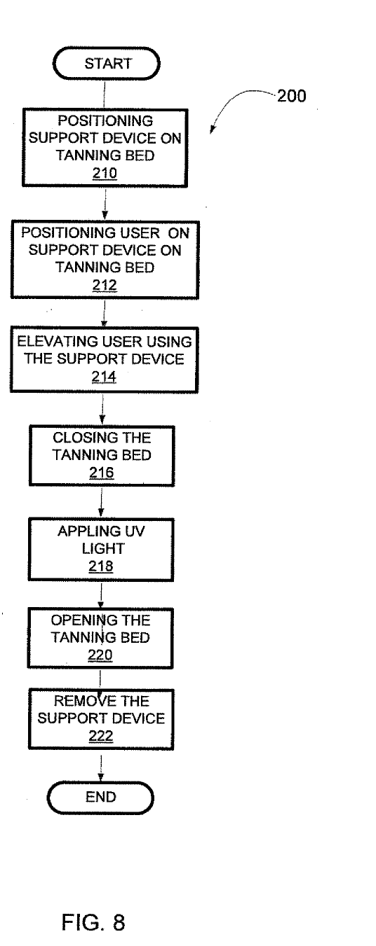

Referring now to FIG. 8, a method for tanning using a tanning bed is illustrated. The method, generally designated 200, comprises positioning a support device (similar to those discussed previously) on a tanning bed (step 210). Numerous tanning beds may be adapted for use with the present invention and are not limited to the description and illustration provided herein. Personal experience, instruction, marked lines, and the like may achieve positioning the device.

In the illustrated embodiment, the user is positioned on the support device (step 212). In one embodiment, the user lies on his/her back on the lower “clam-shell” of the tanning bed one the support device. The width or depth of the device 10 elevates the user's buttocks above the surface of the tanning bed, such that only the user's feet or ankles and shoulder blades or head contact the surface of the tanning bed (step 214). This provides a slight gap for evenly tanning the bottom side of the user.

The upper chamber of the tanning is lowered, closing the tanning bed (step 216). The tanning session begins by application of UV light (step 218). Upon termination of the tanning session, the tanning bed is opened (step 220). In the illustrated embodiment, the device is removed, allowing for easy cleaning, sterilization (e.g., using steam, high UV light, cleaning products, etc.), and re-use by the user or other individuals (step 222). Alternatively, the device may be thrown away or disposed of. In another embodiment, the device is fixed to the tanning bed.

While the embodiments of the invention disclosed herein are presently considered to be preferred, various changes and modifications can be made without departing from the spirit and scope of the invention. For example, the device configuration is not limited to any particular design, shape, geometry, and the like. Additionally, the device may include a house defining a cavity adapted to be filled with a suitable material Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

We claim:

1. A method for tanning using a tanning bed, comprising:
a) positioning a support device on at least one UV emitting surface of the tanning bed;
b) positioning a tailbone region of a user on said support device;
c) elevating said user such that said user's feet and shoulders contact said first surface based on said positioning; and
d) applying ultraviolet light to said user, initiating a tanning session.

2. The tanning method of claim 1 wherein step b) comprises receiving said user's buttocks in a recess formed in said support device.

3. The tanning method of claim 1 comprising step e) terminating said tanning session.

4. The tanning method of claim 3 wherein step e) comprises terminating said application of ultraviolet light and opening the tanning bed.

5. The tanning method of claim 3 wherein step e) comprises removing said support device from said at least one surface.

6. The tanning method of claim 5 wherein step e) comprises sterilizing said support device.

7. The tanning method of claim 5 wherein step e) comprises disposing of said support device.

8. The tanning method of claim 1 wherein said support device comprises:
a first surface defining a first peripheral edge;
a second surface opposite said first surface, said second surface defining a second peripheral edge;
a third surface defining two opposing peripheral edges, at least one of said two opposing peripheral edges contacting said first peripheral edge and the other of said two opposing peripheral edges contacting said second peripheral edge; and
a recess formed in at least one of said first and second surfaces and configured to be placed under said tailbone region of said user and receive said user's buttocks.

9. A tanning bed system, comprising:
a tanning bed;
a body support portion comprising:
a first surface defining a first peripheral edge;
a second surface opposite said first surface, said second surface defining a second peripheral edge;
a third surface defining two opposing peripheral edges, at least one of said two opposing peripheral edges contacting said first peripheral edge and the other of said two opposing peripheral edges contacting said second peripheral edge; and
a recess formed in at least one of said first and second surfaces and configured to be placed under a user's tailbone region.

10. The tanning bed system of claim 9 wherein said recess is formed in said first surface.

11. The tanning bed system of claim 9 wherein at least one of said first, second and third surfaces is substantially planar.

12. The tanning bed system of claim 9 comprising a material that is configured not to compress more than half when a force of less than about 500 pounds is applied thereto.

13. The tanning bed system of claim 12 wherein said material is substantially transparent to ultraviolet light.

14. The tanning bed system of claim 12 wherein said material is substantially opaque to ultraviolet light.

15. The tanning bed system of claim 12 wherein said material is selected from a group consisting of plastic, porous material, foam, silicone, and rubber.

16. The tanning bed system of claim 12 wherein said material is configured to be sterilized and reused.

17. The tanning bed system of claim 12 wherein said material is disposable.

18. A support device for use with a tanning bed, comprising:
a substantially planar first surface having at least two sets of opposing ends, said first surface defining a first peripheral edge;
a substantially planar second surface opposite said first surface, said second surface having at least two sets of opposing ends and defining a second peripheral edge, wherein said substantially planar second surface is configured to contact at least one UV emitting surface of the tanning bed;
a third surface defining two opposing peripheral edges, one of said two opposing peripheral edges contacting said first peripheral edge and the other of said two opposing peripheral edges contacting said second peripheral edge; and
a recess formed in said first surface and configured to be placed under a user's tailbone region, enabling said user to recline in the tanning bed and elevating said user such that only said user's feet and shoulders contact said UV emitting surface.

19. The support device of claim 18 wherein said each of said two sets of opposing edges are about 4 inches in length and said two opposing peripheral edges of said third surface are spaced about 2 inches apart.
